(19) United States
(12) Patent Application Publication
(10) Pub. No.: US 2007/0083178 A1
(43) Pub. Date: Apr. 12, 2007

(54) Feminine pad system

(76) Inventor: Valentina M. Nash, Las Vegas, NV (US)

Correspondence Address: WEISS & MOY PC, 4204 NORTH BROWN AVENUE, SCOTTSDALE, AZ, 85251, US

(21) Appl. No.: 11/244,853

(22) Filed: Oct. 7, 2005

(51) Int. Cl. A61F 13/15

(52) U.S. Cl. 604/385.02; 206/440

(57) ABSTRACT

A feminine pad system comprises a container holding a plurality of differently-sized feminine pads within a single container. In this fashion, a woman can, during a single menstrual period, use different-sized pads from a single container, without the need to purchase and store multiple pad containers.

### FIELD OF THE INVENTION

This invention relates generally to feminine pads and, more particularly, to a feminine pad system wherein a plurality of pad sizes are provided within a single container, so as to accommodate the variability of blood flow within a woman's menstrual period.

### BACKGROUND OF THE INVENTION

On average, menstrual flow occurs every 28 days and lasts about 4 days. It produces a total blood loss of 60 to 250 milliliters (4 tablespoons to about 1 cup). However, for most women, menstrual flow is not consistent. Typically, a woman experiences relatively less blood flow at the beginning of her period, heavier blood flow in the middle, and then progressively lighter blood flow toward the end.

Feminine pads come in different sizes to accommodate different rates of blood flow. In general, this includes thick pads for heavy days, medium pads for medium days, and light pads for lighter days. These are sold separately, typically in containers containing a single size of pad. However, the same woman, during a single menstrual period, may require two or even three sizes of pads. This requires a woman to purchase and store more than one size of pad. This produces waste and inconvenience.

A need therefore exists for a pad system that accommodates the variability of menstrual flow within a woman's period, by providing at least two and preferably more than two sizes of feminine pads within a single container. The present invention satisfies these needs and provides other, related, advantages.

### SUMMARY OF THE INVENTION

In accordance with an embodiment of the present invention, a feminine pad system is disclosed. The system comprises, in combination: a container; a first plurality of feminine pads located in the container; wherein the first plurality of feminine pads comprise a first pad size; and a second plurality of feminine pads located in the container; wherein the second plurality of feminine pads comprise a second pad size that is different from the first pad size.

In accordance with another embodiment of the present invention, a feminine pad system is disclosed. The system comprises, in combination: a container a first plurality of feminine pads located in the container; wherein the first plurality of feminine pads comprise a first pad size; a second plurality of feminine pads located in the container; wherein the second plurality of feminine pads comprise a second pad size that is different from the first pad size; and a third plurality of feminine pads located in the container; wherein the third plurality of feminine pads comprise a third pad size that is different from the first pad size and the second pad size; wherein the first pad size is thick, the second pad size is thin, and the third pad size is medium; wherein the container comprises an upper row holding a plurality of feminine pads and a lower row holding a plurality of feminine pads; and wherein the first row holds the first plurality of feminine pads and wherein the second row holds each of the second plurality of feminine pads and the third plurality of feminine pads.

### BRIEF DESCRIPTION OF THE DRAWINGS

FIG. 1 is a side, cross-sectional view of a feminine pad system consistent with an embodiment of the present invention.

FIG. 2 is a top view of the first (upper) row of the container shown in FIG. 1.

FIG. 3 is a top view of the second (lower) row of the container shown in FIG. 1.

### DETAILED DESCRIPTION OF THE PREFERRED EMBODIMENTS

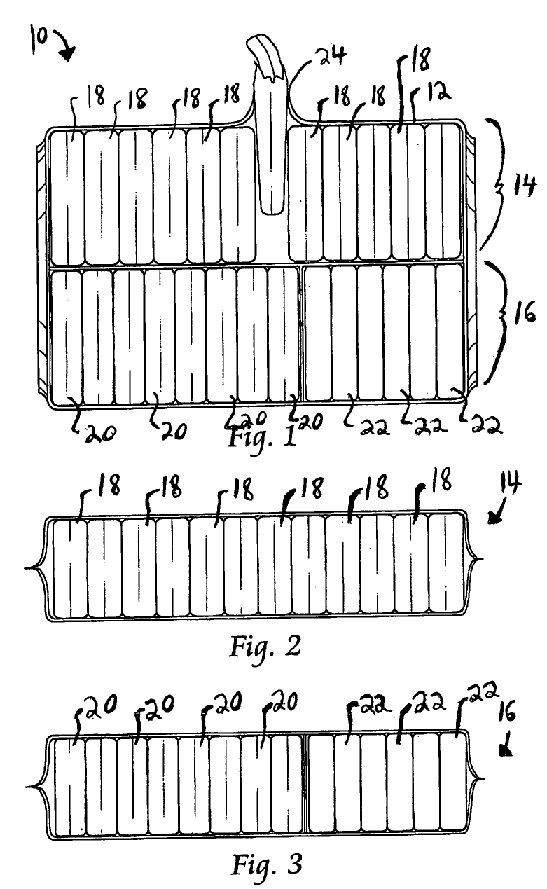

Referring first to FIG. 1, a feminine pad system 10 consistent with an embodiment of the present invention is shown. In this embodiment, the system 10 includes a container 12 holding a plurality of different-sized feminine napkins. In this embodiment, the container 12 is organized so as to have a first/upper row 14 and a second/lower row 16.

It should be noted that the container 12 could be configured in other ways, without departing from the spirit or scope of the present invention. For example, instead of rows, it may be possible to provide dividers that will separate differently-sized pads within a single row. It may be desired to provide three, or even more than three rows. It may also be desired to simply provide a plurality of differently-sized pads within a single container, without a divider or row system to separate them.

It should also be noted that it may be desired to differently color the packaging for the individual feminine pads, based on size, so as to facilitate differentiation between different-sized pads. In addition or in the alternative, it may be desired to label the packaging for the individual feminine pads with the pad size.

Referring now to FIGS. 1-2, in this embodiment, a plurality of thick pads 18 are provided on the first/upper row 14. Referring to FIGS. 1 and 3, on the second/lower row 16, there are provided a plurality of medium pads 20 and a plurality of thin pads 22.

FIG. 1 illustrates the removal of a thick pad 18 through an opening 24 in the container 12. It may be desired to provide only a single opening 24, or a plurality of openings. Where a plurality of openings are provided, it may be desired to provide at least one opening for each pad size contained within the container 12, with the opening to be located proximate the pads which are to be removed therethrough.

As shown in FIG. 1, the opening 24 could be a dedicated opening for use solely for the removal of thick pads 18, with a second opening (not shown) to be provided for the removal of medium pads 20, and third opening (not shown) to be provided for the removal of thin pads 22. Where a plurality of openings are provided, it may be desired to label them with respect to the pad size to be removed therethrough, to permit the user to more readily identify the correct opening for the removal of the desired pad.

While various embodiments of the present invention are shown and described herein, it should be understood that various may be possible without departing from the spirit or scope of the present invention. For example, it may be desired to provide only two sizes of pads within the container 12, or it may be desired to provide more than three sizes. It may also be desired to provide a variety of feminine hygiene products of a type that a single user may desire during a single period, such as tampons, panty shields, and the like—in addition to one or more sizes of feminine pads.

I claim:

1. A feminine pad system comprising:
a container;
a first plurality of feminine pads located in the container;
wherein the first plurality of feminine pads comprise a first pad size; and
a second plurality of feminine pads located in the container;
wherein the second plurality of feminine pads comprise a second pad size that is different from the first pad size.

2. The system of claim 1 wherein the first pad size is thick.

3. The system of claim 2 wherein the second pad size is thin.

4. The system of claim 1 further comprising:
a third plurality of feminine pads located in the container;
wherein the third plurality of feminine pads comprise a third pad size that is different from the first pad size and the second pad size.

5. The system of claim 4 wherein the first pad size is thick, the second pad size is thin, and the third pad size is medium.

6. The system of claim 1 wherein the container comprises an upper row holding a plurality of feminine pads and a lower row holding a plurality of feminine pads.

7. The system of claim 4 wherein the container comprises an upper row holing a plurality of feminine pads and a lower row holding a plurality of feminine pads.

8. The system of claim 7 wherein the first row holds the first plurality of feminine pads and wherein the second row holds each of the second plurality of feminine pads and the third plurality of feminine pads.

9. The system of claim 8 wherein the second plurality of feminine pads is held on a first side of the second row and wherein the third plurality of feminine pads is held on a second side of the second row.

10. A feminine pad system comprising:
a container;
a first plurality of feminine pads located in the container;
wherein the first plurality of feminine pads comprise a first pad size;
a second plurality of feminine pads located in the container;
wherein the second plurality of feminine pads comprise a second pad size that is different from the first pad size; and
a third plurality of feminine pads located in the container;
wherein the third plurality of feminine pads comprise a third pad size that is different from the first pad size and the second pad size;
wherein the first pad size is thick, the second pad size is thin, and the third pad size is medium;
wherein the container comprises an upper row holding a plurality of feminine pads and a lower row holding a plurality of feminine pads; and
wherein the first row holds the first plurality of feminine pads and wherein the second row holds each of the second plurality of feminine pads and the third plurality of feminine pads.

11. The system of claim 10 wherein the second plurality of feminine pads is held on a first side of the second row and wherein the third plurality of feminine pads is held on a second side of the second row.
